(19) United States
(12) Patent Application Publication
(10) Pub. No.: US 2026/0234098 A1
(43) Pub. Date: Aug. 13, 2026

(54) PROCESS FOR PREPARING ESTERAMIDE COMPOUNDS

(71) Applicant: SPECIALTY OPERATIONS FRANCE, Lyon (FR)

(72) Inventors: Olivier Back, Lyon (FR); Julien Rabih Rachet, Vernaison (FR)

(73) Assignee: SPECIALTY OPERATIONS FRANCE, Lyon (FR)

(21) Appl. No.: 19/475,052

(22) PCT Filed: Apr. 8, 2024
(86) PCT No.: PCT/EP2024/059461

§ 371 Date: Oct. 13, 2025

(30) Foreign Application Priority Data
Apr. 13, 2023 (EP) 23167672.7

(51) Int. Cl. C07C 231/02

(52) U.S. Cl. CPC C07C 231/02

(57) ABSTRACT

A process for preparing esteramide compounds of formula (IV)

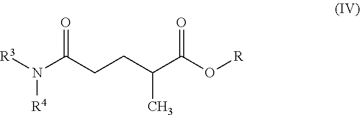

wherein R is an alkyl group; and R³ and R⁴ are identical or different, and are each selected from among saturated or unsaturated, linear or branched, optionally cyclic, optionally aromatic, optionally substituted hydrocarbon-based groups having an average number of carbon atoms ranging from 1 to 36, with the proviso that R³ and R⁴ may optionally together form a ring member, that is optionally substituted and/or that optionally contains a heteroatom; and said process comprising the steps of: i) dimerization of an alkyl acrylate of formula (I)

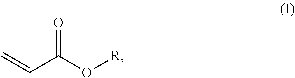

to obtain a dimer of formula (II)

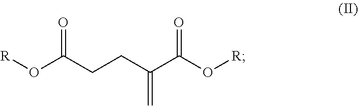

ii) hydrogenation of the dimer of formula (II) to obtain a compound of formula (III)

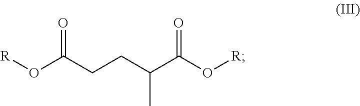

and iii) reacting the compound of formula (III) with an amine of formula HNR³R⁴.

This application claims priority to the application filed on 2023 Apr. 13 in Europe with Nr 23167672.7, the whole content of this application being incorporated herein by reference for all purposes.

### FIELD OF THE INVENTION

The present invention relates to a process for preparing esteramide compounds.

### TECHNICAL BACKGROUND

Esteramide compounds are useful solvents/coalescing agents for a variety of phytosanitary, cleaning, degreasing, stripping, lubricating, coating and pigment/ink compositions. Corresponding compositions are described in U.S. Pat. No. 8,735,324, which also describes a process for preparing esteramide compounds by reacting a diester compound with an amine.

In industrial processes currently used, the required diesters are obtained by reacting, for example a mixture of adiponitrile, 2-methyl glutaronitrile, and 2-ethylsuccinonitrile a byproduct of the synthesis of adiponitrile, with water under acidic conditions to generate the diacid intermediate mixture which is then esterified in a second step with methanol to obtain a mixture of dimethyl 2-methylglutarate, dimethyl 2-ethyl succinate and dimethyl adipate, as for example described in U.S. Pat. No. 9,267,015.

This synthesis approach has, however, the disadvantage that a mixture of diacid esters and, thus, esteramide compounds is obtained. Consequently, if a pure esteramide compound is desired, for example pure methyl-5-(dimethylamino)-2-methyl-5-oxopentanoate, cumbersome purification steps are required. Besides, if the mixture is used as such, this makes its recycling more difficult.

### SUMMARY OF THE INVENTION

It is therefore an object of the present invention to provide a process, which yields esteramide compounds at high purity. Further aims are to provide a process for preparing esteramide compounds at good yield, preferably starting from easily accessible starting compounds. Still further aims are to provide a process for preparing esteramide compounds generating low amounts of wastes and aqueous effluents and displaying a very good atom economy.

It has now been found that these and other problems can be solved by the process of the present invention. The present invention relates to a process for preparing an esteramide compound of formula (IV)

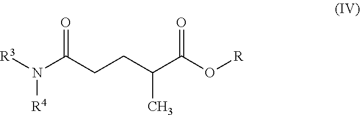

wherein
R is an alkyl group;
R³ and R⁴ are identical or different, and are each selected from among saturated or unsaturated, linear or branched, optionally cyclic, optionally aromatic, optionally substituted hydrocarbon-based groups having an average number of carbon atoms ranging from 1 to 36, with the proviso that R³ and R⁴ may optionally together form a ring member, that is optionally substituted and/or that optionally contains a heteroatom;
said process comprising the steps of:
i) dimerization of an alkyl acrylate of formula (I)

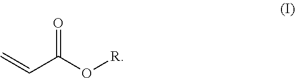

wherein R is defined as above
to obtain a dimer of formula (II)

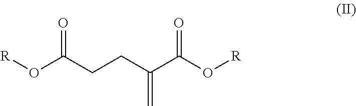

wherein R is defined as above;
ii) hydrogenation of the dimer of formula (II) to obtain a compound of formula (III)

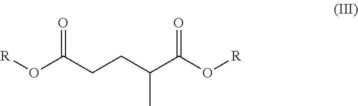

wherein R is defined as above; and
iii) reacting the compound of formula (III) with an amine of formula HNR³R⁴, wherein R³ and R⁴ are defined as above.

The present invention is based on the recognition that the diester intermediates in the prior art synthesis of esteramide compounds can be obtained in high purity by an efficient process for producing an alkyl acrylate dimer, which can readily be hydrogenated. Thus accordingly the esteramide compounds obtained after amidification of such diester intermediates can be prepared at high purity and good yield from readily accessible starting compounds, such as alkyl acrylates and in particular methyl acrylate.

### DETAILED DESCRIPTION OF THE INVENTION

According to the present invention the term “about” means±10% of the specified numeric value, preferably ±5% and most preferably ±2%.

The present invention relates to a process for preparing an esteramide compound of formula (IV)

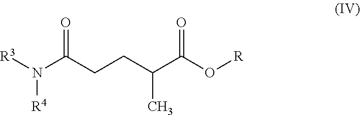

wherein
R is an alkyl group;
R³ and R⁴ are identical or different, and are each selected from among saturated or unsaturated, linear or branched, optionally cyclic, optionally aromatic, optionally substituted hydrocarbon-based groups having an average number of carbon atoms ranging from 1 to 36, with the proviso that R³ and R⁴ may optionally together form a ring member, that is optionally substituted and/or that optionally contains a heteroatom;
said process comprising the steps of:
i) dimerization of an alkyl acrylate of formula (I)

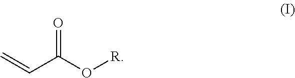

wherein R is defined as above
to obtain a dimer of formula (II)

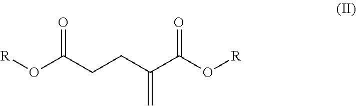

wherein R is defined as above;
ii) hydrogenation of the dimer of formula (II) to obtain a compound of formula (III)

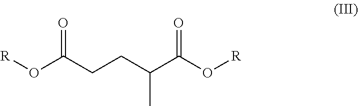

wherein R is defined as above; and
iii) reacting the compound of formula (III) with an amine of formula HNR³R⁴, wherein R³ and R⁴ are defined as above.

In one embodiment, the dimerization step i) is conducted in the presence of a catalyst of formula (V)

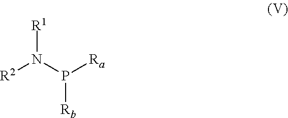

wherein
R¹ and R² are identical or different, and are either aliphatic groups or form together with the N atom a heteroaliphatic cycle;
Rₐ is a hydrocarbyl group;
R_{b} is either an aliphatic group or NR⁵R⁶ with R⁵ and R⁶ being identical or different, and being either aliphatic groups or forming together with the N atom a heteroaliphatic cycle.

In a further embodiment, the dimerization step i) is conducted in the presence of a compound A being a tertiary alcohol or a silanol, preferably a tertiary alcohol, such as tert-butanol, tert-amyl alcohol or pinacol and more preferably tert-butanol.

Preferably, in the process for producing a dimer according to formula (II) as defined herein, the molar ratio [compound A]/[alkyl acrylate according to formula (I)] is selected from about 4:1 to about 0.01:1, preferably from about 2:1 to about 0.1:1 and more preferably from about 0.5:1 to about 0.1:1, and notably from about 0.5:1 to about 0.2:1.

Preferably, in the process for producing a dimer according to formula (II) as defined herein, R is a C₁-C₁₈, more preferably a C₁-C₈alkyl, still more preferably a C₁-C₄ alkyl.

Preferably, in the process for producing a dimer according to formula (II) as defined herein, R is a methyl group, ethyl group, propyl group, isopropyl group, n-butyl group, sec-butyl group, isobutyl group, tert-butyl group, pentyl group, hexyl group, 2-ethylhexyl group, octyl group, decyl group, dodecyl group, t-dodecyl group, tetradecyl group, hexadecyl group or an octadecyl group, more preferably, a methyl group, ethyl group, isopropyl group, butyl group or 2-ethylhexyl group, still more preferably a methyl group, ethyl group, isopropyl group or a butyl group, most preferably a methyl group.

Preferably, in the process for producing a dimer according to formula (II) as defined herein, R¹ and R² are identical linear or branched alkyl groups comprising from 1 to 6 carbon atoms, more preferably from 1 to 3 carbon atoms, most preferably ethyl.

Preferably, in the process for producing a dimer according to formula (II) as defined herein, R¹ and R² form together with the N atom a heteroaliphatic cycle comprising from 3 to 5 carbon atoms, preferably 4 carbon atoms.

Preferably, in the process for producing a dimer according to formula (II) as defined herein, Rₐ is either an aromatic or an aliphatic group, more preferably an aromatic group, still more preferably selected from phenyl, tolyl, xylyl, mesityl, duryl, pentamethylphenyl, 2,6-diisopropylphenyl, tert-butylphenyl, ditert-butylphenyl, methoxyphenyl, dimethoxyphenyl, methoxytolyl, methylenedioxyphenyl, biphenyl, nitrophenyl, halogen substituted phenyl, trifluoromethylphenyl, naphtyl, pyridyl, furyl, pyrrolyl, thiophenyl, 2-indolyl, benzofuryl and all their position isomers.

Preferably, Rₐ is selected from phenyl; ortho-, meta- or para-tolyl; xylyl including all position isomers such as: 2,3-dimethylphenyl, 2,4-dimethylphenyl, 2,5-dimethylphenyl, 2,6-dimethylphenyl, 3,4-dimethylphenyl and 3,5-dimethylphenyl; 3-methyl-4-methoxyphenyl, 2-methyl-4-methoxyphenyl, 2-methyl-3-methoxyphenyl, 4-methyl-3-methoxyphenyl, 5-methyl-3-methoxyphenyl, 6-methyl-3-methoxyphenyl 2-methoxy-3-methylphenyl, 2-methoxy-4-methylphenyl, 2-methoxy-5-methylphenyl, 2-methoxy-6-methylphenyl; mesityl including all position isomers such as: 2,3,4-trimethylphenyl, 2,3,5-trimethylphenyl, 2,3,6-trimethylphenyl, 2,4,5-trimethylphenyl, 2,4,6-trimethylphenyl and 3,4,5-trimethylphenyl; duryl including all position isomers such as: 2,3,4,5-tetramethylphenyl, 2,3,4,6-tetramethylphenyl and 2,3,5,6-tetramethylphenyl; pentamethylphenyl, 2,6-diisopropylphenyl; ortho-, meta- or para-tert-butylphenyl; 2,3-di-tert-butylphenyl, 2,4-di-tert-butylphenyl, 2,5-di-tert-butylphenyl, 2,6-di-tert-butylphenyl, 3,4-di-tert-butylphenyl and 3,5-di-tert-butylphenyl; ortho-, meta- or para-methoxyphenyl; ortho-, meta- or para-chlorophenyl; 2,3-dimethoxyphenyl, 2,4-dimethoxyphenyl, 2,5-dimethoxyphenyl, 2,6-dimethoxyphenyl, 3,4-dimethoxyphenyl and 3,5-dimethoxyphenyl; 2,3-methylenedioxyphenyl, 3,4-methylenedioxyphenyl; ortho-, meta- or para-nitrophenyl; ortho-, meta- or para-biphenyl; ortho-, meta- or para-trifluoromethylphenyl, ortho-, meta- or para-fluorophenyl; 1- or 2-naphtyl; 2-pyridyl, 3-pyridyl or 4-pyridyl; 2-furyl, 3-furyl; 1-pyrrolyl, 2-pyrrolyl or 3-pyrrolyl; 2-thiophenyl, 3-thiophenyl; 2-indolyl, 3-indolyl, 2-benzofuryl and 3-benzofuryl; preferably phenyl; ortho-, meta- or para-tolyl; or xylyl and its isomer positions.

Preferably, in the process for producing a dimer according to formula (II) as defined herein, R_{b} is NR⁵R⁶ with R⁵ and R⁶ being identical or different, and being either an aliphatic group or forming together with the N atom a heteroaliphatic cycle, more preferably R⁵ and R⁶ are identical linear or branched alkyl groups comprising from 1 to 6 carbon atoms, still more preferably from 1 to 3 carbon atoms, most preferably ethyl.

Preferably, in the process for producing a dimer according to formula (II) as defined herein, Rₐ is a phenyl, R¹ and R² are ethyl and R_{b} is NR⁵R⁶ with R⁵ and R⁶ being ethyl.

Preferably, in the process for producing a dimer according to formula (II) as defined herein, the catalyst according to formula (V) is a compound selected from the group consisting of the compounds according to formulae (VIII) to (XIV):

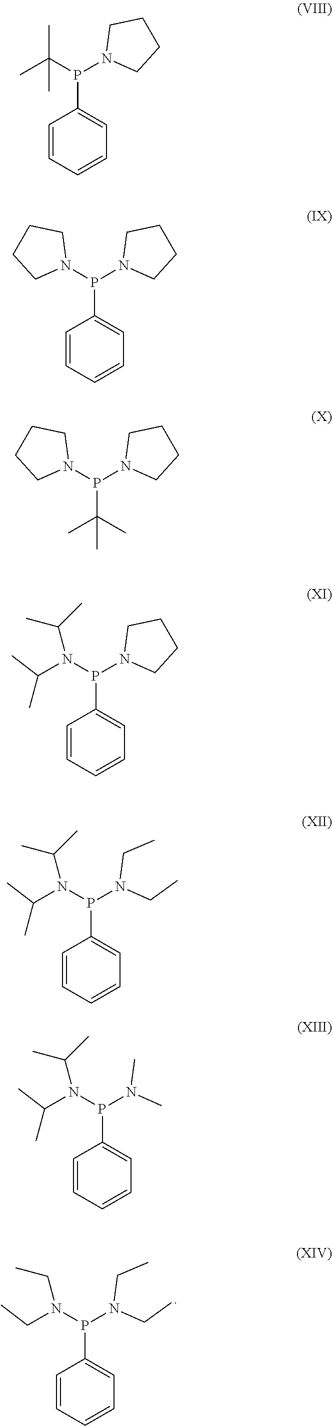

More preferably, in the process for producing a dimer according to formula (II) as defined herein, the catalyst according to formula (V) is a compound selected from the group consisting of compounds according to formulae (IX) and (XI) to (XIV), still more preferably selected from the group consisting of compounds according to formulae (XI), (XII) and (XIV), even more preferably selected from the group consisting of compounds according to formulae (XI) and (XIV), most preferably the catalyst according to formula (V) is the compound according to formula (XIV).

Preferably, in the process for producing a dimer according to formula (II) as defined herein, the step i) of dimerization is performed in an organic solvent, more preferably an aprotic solvent, still more preferably selected from tetrahydrofuran (THF), 2-methyltetrahydrofuran (MeTHF), toluene, xylene, anisole, diethyl ether, tert-butyl methyl ether (MTBE), dichloromethane (DCM), chloroform, 1,4-dioxane, pentane, cyclopentane, hexane, cyclohexane, methylcyclohexane, benzene and acetonitrile, even more preferably MeTHF, anisole and toluene, most preferably MeTHF and anisole.

Preferably, in the process for producing a dimer according to formula (II) as defined herein, the dimerization step i) is performed at a temperature ranging from about 20° C. to about 120° C., more preferably about 20° C. to about 80° C., still more preferably about 25° C. to about 60° C., most preferably from about 30° C. to about 60° C.

Preferably, in the process for producing a dimer according to formula (II) as defined herein, in step i) the catalyst according to formula (V) is used with a catalyst loading of from 0.20 mol % to 1.00 mol % with respect to the alkyl acrylate according to formula (I), more preferably of from about 0.25 mol % to about 0.90 mol %, still more preferably of from about 0.30 mol % to about 0.90 mol %, even more preferably of from about 0.30 mol % to about 0.80 mol %, even still more preferably of from about 0.30 mol % to about 0.70 mol %, even still more preferably of from about 0.30 mol % to about 0.60 mol %, most preferably of from about 0.30 mol % to 0.50 mol %.

Preferably, in the process for producing a dimer according to formula (II) as defined herein, the dimerization step i) is carried out in anhydrous conditions and in the absence of oxygen.

Preferably, the process for producing a dimer according to formula (II) as defined herein, further comprises an initial step 0) of preparation of the catalyst according to formula (V) by reacting a compound according to formula (VI)

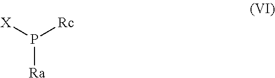

wherein
X is a chloride, a bromide or an iodide, preferably chloride;
Rₐ is a hydrocarbyl group;
R_{c} is either X (for the case of catalysts of formula (V) wherein R_{b} is NR⁵R⁶ as defined above) or R_{b} (for the case of catalysts of formula (V) wherein R_{b} is an aliphatic group);
with
an amine of formula R¹R²NH, with R¹ and R² being as defined herein when R_{c} is R_{b} or
both an amine of formula R¹R²NH and an amine of formula R⁵R⁶NH, with R⁵ and R⁶ being as defined herein when R_{c} is X.

Preferably, in the process for producing a dimer according to formula (II) as defined herein, step 0) and step i) are consecutive steps performed without isolation of the catalyst after step 0).

Preferably, in the process for producing a dimer according to formula (II) as defined herein, R_{c} is X.

Preferably, in the process for producing a dimer according to formula (II) as defined herein, the step 0) is performed in an organic solvent, more preferably an aprotic solvent, still more preferably selected from tetrahydrofuran (THF), 2-methyltetrahydrofuran (MeTHF), toluene, xylene, anisole, diethyl ether, tert-butyl methyl ether (MTBE), dichloromethane (DCM), chloroform, 1,4-dioxane, pentane, cyclopentane, hexane, cyclohexane, methylcyclohexane, benzene and acetonitrile, even more preferably MeTHF, anisole and toluene, most preferably MeTHF and anisole.

Preferably, in the process for producing a dimer according to formula (II) as defined herein, step 0) is performed at a temperature ranging from about 20° C. to about 100° C., preferably about 20° C. to 80° C., more preferably about 25° C. to 60° C., most preferably at a temperature of about 40° C.

Preferably, in the process for producing a dimer according to formula (II) as defined herein, step 0) is performed through the slow addition of the reactant of formula (VI) to a solution of the amine R¹R²NH in the aprotic solvent where the amine is used in an amount equal to or greater than 2 equivalents with respect to the reactant of formula (VI) when R_{c} in (VI) is R_{b} and R_{b} is an aliphatic group. Step (0) can also be performed through the slow addition of the reactant of formula (VI) to a solution containing both the amine R¹R²NH and R⁵R⁶NH in the aprotic solvent where the total amount of the amines is equal or greater than 4 equivalents with respect to the reactant for formula (VI) when R_{c} in (VI) is X.

Preferably, in the process for producing a dimer according to formula (II) as defined herein, step 0) is carried out in anhydrous conditions and in the absence of oxygen.

Preferably, in the process for producing a dimer according to formula (II) as defined herein, step 0) comprises a filtration step to remove ammonium halide by-products formed before performing step i).

Moreover, the present invention includes a step of producing a compound according to formula (III), comprising the process for producing a dimer according to formula (II) as defined herein, followed by a step ii) of hydrogenation of the dimer according to formula (II) obtained in the step of dimerization preferably using H₂ and a hydrogenation catalyst, such as Pd based catalysts, for example Pd/C, Pd/Al₂O₃, Pd/SiO₂, Ru based catalysts, for example Ru/C, Pt based catalysts such as Pt/C, Ni based catalysts, such as supported nickel or Raney nickel catalysts, Co based catalysts, such as supported cobalt or Raney cobalt, Rh based catalysts, such as Rh/C, Ir based catalysts, such as Ir/C, preferably Pd/C or Raney nickel, preferably Pd/C, to obtain a compound according to formula (III)

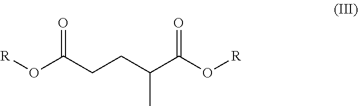

wherein R is as defined herein.

Furthermore, the process of the present invention includes a step iii) of reacting the compound of formula (III) with an amine of formula HNR³R⁴, wherein
R³ and R⁴ are identical or different, and are each selected from among saturated or unsaturated, linear or branched, optionally cyclic, optionally aromatic, optionally substituted hydrocarbon-based groups having an average number of carbon atoms ranging from 1 to 36, with the proviso that R³ and R⁴ may optionally together form a ring member, that is optionally substituted and/or that optionally contains a heteroatom.

The R³ and R⁴ groups, which are identical or different, may especially be groups chosen from C₁-C₁₂ alkyl, aryl, alkaryl or arylalkyl groups or the phenyl group. The R³ and R⁴ groups may optionally be substituted, in particular by hydroxyl groups.

The R³ and R⁴ groups, which are identical or different, may especially be chosen from methyl, ethyl, propyl (n-propyl), isopropyl, n-butyl, isobutyl, n-pentyl, amyl, isoamyl, hexyl, cyclohexyl or 2-hydroxyethyl groups. The R³ and R⁴ groups may also be such that they form, together with the nitrogen atom, a morpholine, piperazine, pyrrolidine or piperidine group. According to particular embodiments, R³=R⁴=methyl, or R³=R⁴=ethyl, or R³-R⁴=2-hydroxyethyl. Good results are obtained when R³=R⁴=methyl.

During step iii), preferably from 0.7 to 1.5 mol, for example 0.8 to 1.2 mol, preferably from 0.9 to 1.1 mol, preferably about 1 mol of amine is used per mole of hydrogenated dimer of formula (III). It is advantageous to operate with a slight excess such as an excess of at least 1.05 mol of amine per mole of dimer, for example from 1.05 to 1.1 mol of amine per mole of dimer.

Step iii) may be carried out in solution, for example in solution in a solvent such as toluene or an alcohol. It is however preferred to operate in a non-aqueous solution, avoiding any presence of water. It is possible, during this step, to gradually eliminate, for example, the alcohol that is co-produced in order to favor the reaction. The elimination may be accompanied by an elimination of the solvent, for example using an azeotrope. After separation of the alcohol, the solvent eliminated may be reintroduced into the process.

Step iii) is preferably carried out in the presence of a catalyst, in particular a basic type catalyst. It is possible, for example, to use methylates such as MeONa, carbonates such as K₂CO₃ or Na₂CO₃, or titanates such as titanium tetraethoxide.

The esteramide compounds obtained by the process according to the invention can for example be used as solvents, in particular as solvents for preparing agrochemical formulations.

Should the disclosure of any patents, patent applications, and publications which are incorporated herein by reference conflict with the description of the present application to the extent that it may render a term unclear, the present description shall take precedence.

### EXAMPLES

### Example 1: Dimerization of Methyl Acrylate

Analytically pure dimethyl 2-methyleneglutarate was obtained following the general protocol described in point A below, and using the specific conditions described in point B below, all products obtained being mixed in the end to produce one single lot.

### A. General Protocol:

All the reactions were conducted in carefully dried vessels and under an inert argon atmosphere. Methyl acrylate and tert-butanol were dried using 4 A molecular sieves and tert-butanol was distilled under argon prior to each reaction.

### A1. Catalyst Preparation

Bis(diethylamino)phenylphosphine was synthesized by adding progressively a dichlorophenylphosphine (1 equivalent) in 2-methyltetrahydrofuran (Me-THF) solution to a diethylamine (4 equivalents) solution in Me-THF under stirring (1400 rpm) over 1 hour while keeping the temperature of the reaction medium below 40° C. (exothermic reaction). Upon dichlorophenylphosphine addition, there was formation of a white precipitate corresponding to the ammonium chloride salt by-product (in this case diethylammonium chloride). At the end of the addition, the mixture was then allowed to stir at ambient temperature and the reaction progress was monitored thanks to NMR.

After bis-(diethylamino)phenylphosphine formation completion which requires 1 h of stirring at room temperature after the dichlorophenylphosphine addition, the mixture was filtrated.

(diisopropylamino)pyrrolidinophenylphosphine in solution in 2-methyltetrahydrofuran (Me-THF) was synthesized in a similar way except that:
diisopropylamine (3 equivalents) was used instead of diethylamine
the final reaction mixture was further allowed to stir at room temperature and 1 equivalent of pyrrolidine was added to the reaction mixture which was allowed to stir at room temperature for an additional hour in order to complete bis-(amino)phosphine formation.

### A2. Dimerization

Bis(diethylamino)phenylphosphine or (diisopropylamino)pyrrolidinophenylphosphine in solution in 2-methyltetrahydrofuran (Me-THF), was introduced into a 500 mL double jacketed reactor equipped with a temperature probe, a condenser and a mechanical stirrer (propeller with four inclined plows) and baffles, and containing:
distillated tert-butanol
methyl acrylate.

The reaction progress was monitored thanks to 1H NMR. The conversion of methyl acrylate was estimated by 1H NMR thanks to the integration of the methylene protons of the products and the methylene protons in the starting methyl acrylate.

At the end of the reaction, the volatiles (t-BuOH, Me-THF and unconverted methyl acrylate) were removed under vacuum. The desired product (dimethyl 2-methyleneglutarate) was then distilled under vacuum (160° C., 15 mbar) to afford analytically pure product. High boiling point by-products (methyl acrylate oligomers) remained in the distillation vessel.

### B. Specific Conditions

a) Bis(diethylamino)phenylphosphine catalyzed (initial 0.7 mol % dichlorophenylphosphine precursor with respect to methyl acrylate) methyl acrylate dimerization in tert-butanol (1:4 v/v t-BuOH:methyl acrylate=0.24 mol(t-BuOH)/mol(Me-Acrylate)), 45° C.—same at 30° C.
b) Bis(diethylamino)phenylphosphine catalyzed (0.7 mol % dichlorophenylphosphine precursor with respect to methyl acrylate) methyl acrylate dimerization in tert-butanol (1:8 v/v t-BuOH:methyl acrylate=0.12 mol(t-BuOH)/mol(Me-Acrylate)), 45° C.—same at 30° C. and 60° C.
c) Bis(diethylamino)phenylphosphine catalyzed (0.9 mol % dichlorophenylphosphine precursor with respect to methyl acrylate) methyl acrylate dimerization in tert-butanol (1:4 v/v t-BuOH:methyl acrylate=0.24 mol(t-BuOH)/mol(Me-Acrylate)), 30° C.
d) (diisopropylamino)pyrrolidinophenylphosphine catalyzed (0.4 mol % dichlorophenylphosphine precursor with respect to methyl acrylate) methyl acrylate dimerization in tert-butanol (1:8 v/v t-BuOH:methyl acrylate=0.12 mol(t-BuOH)/mol(Me-Acrylate)), 60° C.

### Example 2: Dimethyl 2-Methyleneglutarate Catalytic Hydrogenation to Dimethyl 2-Methylglutarate

The substrate dimethyl 2-methyleneglutarate produced in Example 1 was first added into a 100 mL autoclave reactor equipped with a mechanical stirrer (Rushton turbine) followed by the addition of the Pd/C (3%) catalyst (powder, 51% moisture content, 1 g wet corresponding to 0.49 g dry, 1.73 wt % with respect to the substrate). The reactor was then tightly sealed and was purged 3 times with 20 bar of nitrogen followed by 3 times with 5 bar of hydrogen. The reaction mixture was allowed to stir at 1400 rpm and the temperature of the reaction mixture was then set at 25° C. The reaction medium was then allowed to stir at 25° C., 3-6 bar hydrogen pressure (1400 rpm) during 3 hours and hydrogen consumption was followed over time.

At the end of the reaction which was confirmed by the absence of hydrogen consumption, the reaction mixture was allowed to cool down at room temperature, stirring was stopped and the autoclave was depressurized. The reactor was purged with nitrogen, the crude was removed from the reactor and the catalyst has been filtered out and recycled in a second hydrogenation batch conducted this time at 80° C. (3-6 bar H₂ pressure) during 8 hours.

The product dimethyl 2-methylglutarate was obtained after catalyst filtration as a clear liquid and was used as such.

### Example 3: Conversion of dimethyl 2-methylglutarate to methyl-5-(dimethylamino)-2-methyl-5-oxopentanoate

The reaction was carried out in a double-jacket reactor equipped with a condenser, a mechanical stirrer and a temperature probe. Before starting the synthesis operation, the reactor was washed first with DI water (to remove acidity) and then with refluxing dried methanol to remove moisture traces.

Then, the following sequences were applied:
1) 298.39 g (1.71 moles, 1 eq.) of dimethyl 2-methylglutarate from Example 2 was charged at room temperature into the reactor and stirring was started.
2) The reaction mass was then cooled down to 6-9° C. and at the same time the temperature in the condenser was set-up at the same temperature.
3) 85.28 g (1.89 moles, 1.1 eq.) of dimethylamine gas (DMA) was then condensed into the reactor by bubbling it in the liquid phase. The absorption of DMA in dimethyl 2-methylglutarate is exothermic therefor the temperature of the reaction mass had to be kept below 15° C. by adjusting the double-jacket temperature and the DMA gas flow rate.
4) Once the right amount of DMA had been charged into the reactor, the charging line was flushed through with nitrogen.
5) 13.2 g of a solution of sodium methoxide in methanol at 28 wt % concentration (corresponding to 3.70 g of NaOMe, 0.068 mole, 4 mol % with respect to dimethyl 2-methylglutarate) was charged into the reactor over approximately 10 minutes. The mass temperature was controlled as much as possible to avoid DMA boiling (temperature kept below 10° C.).
6) The charging line was then flushed with 20.08 g of dry methanol which is added over 30 minutes.
7) The reaction mixture was then allowed to stir at 50° C. and the reaction progress was followed-up over time thanks to GC analysis.
8) After 2 h30 stirring at 50° C., the concentration of the starting bis-ester in the reaction mass was 1.14 wt % and the concentration of methyl-5-(dimethylamino)-2-methyl-5-oxopentanoate was 91.54 wt %.
9) The reaction mixture was then allowed to cool down at room temperature and discharged from the reactor.
10) The base catalyst was then neutralized by the addition of 4.075 g of H3PO4 (85 wt % aqueous solution) and the phosphate salts that precipitated were separated by filtration.
11) The solid was washed several times with methanol.
12) At this stage some deposit was formed in the filtrate which was filtered again without methanol washing. The solvents (water and methanol) were then removed by vacuum distillation (80° C., 10 mbar) allowing to recover at the end 273.8 g of crude methyl-5-(dimethylamino)-2-methyl-5-oxopentanoate which was again filtered to remove any solids providing 268.49 g of clear liquid.

1. A process for preparing an esteramide compound of formula (IV) 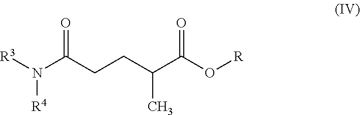
wherein:
R is an alkyl group; and
R³ and R⁴ are identical or different, and are each selected from among saturated or unsaturated, linear or branched, optionally cyclic, optionally aromatic, optionally substituted hydrocarbon-based groups having an average number of carbon atoms ranging from 1 to 36, with the proviso that R³ and R⁴ may optionally together form a ring member, that is optionally substituted and/or that optionally contains a heteroatom;
said process comprising the steps of:
i) dimerization of an alkyl acrylate of formula (I) 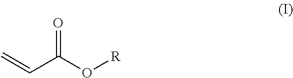
wherein R is an alkyl group
to obtain a dimer of formula (II) 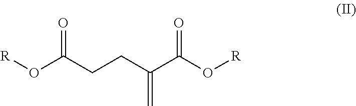
wherein R is an alkyl group;
ii) hydrogenation of the dimer of formula (II) to obtain a compound of formula (III) 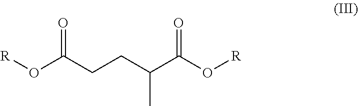
wherein R is an alkyl group; and
iii) reacting the compound of formula (III) with an amine of formula HNR³R⁴, wherein R³ and R⁴ are identical or different, and are each selected from among saturated or unsaturated, linear or branched, optionally cyclic, optionally aromatic, optionally substituted hydrocarbon-based groups having an average number of carbon atoms ranging from 1 to 36, with the proviso that R³ and R⁴ may optionally together form a ring member, that is optionally substituted and/or that optionally contains a heteroatom.

2. The process according to claim 1, wherein the dimerization step i) is conducted in the presence of a catalyst of formula (V) 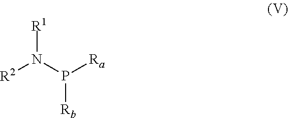
wherein:
R¹ and R² are identical or different, and are either aliphatic groups or form together with an N atom a heteroaliphatic cycle;
Rₐ is a hydrocarbyl group;
R_{b} is either an aliphatic group or NR⁵R⁶ with R⁵ and R⁶ being identical or different, and being either aliphatic groups or forming together with the N atom a heteroaliphatic cycle.

3. The process according to claim 1, wherein the dimerization step i) is conducted in the presence of a compound A being a tertiary alcohol or a silanol.

4. The process according to claim 3, wherein a molar ratio [compound A]/[alkyl acrylate according to formula (I)] is selected from about 4:1 to about 0.01:1.

5. The process according to claim 1, wherein R is a C₁-C₁₈.

6. The process according to claim 2, wherein R¹ and R² are identical linear or branched alkyl groups comprising from 1 to 6 carbon atoms.

7. The process according to claim 2, wherein R¹ and R² form together with the N atom a heteroaliphatic cycle comprising from 3 to 5 carbon atoms.

8. The process according to claim 2, wherein Rₐ is either an aromatic or an aliphatic group.

9. The process according to claim 2, wherein R_{b} is NR⁵R⁶ with R⁵ and R⁶ being identical or different, and being either an aliphatic group or forming together with the N atom a heteroaliphatic cycle.

10. The process according to claim 2, wherein Rₐ is a phenyl, R¹ and R² are ethyl and R_{b} is NR⁵R⁶ with R⁵ and R⁶ being ethyl.

11. The process according to claim 1, wherein the dimerization of step i) is performed in an organic solvent.

12. The process according to claim 1, wherein the dimerization step i) is performed at a temperature ranging from about 20° C. to about 120° C.

13. The process according to claim 2, wherein R³ and R⁴ are identical or different, and are selected from among C₁-C₁₂ alkyl, aryl, alkaryl and arylalkyl which each may optionally be substituted.

14. The process according to claim 1, wherein step iii) is conducted in an aqueous or non-aqueous solution.

15. The process according to claim 1, wherein step iii) is conducted in the presence of a catalyst.

16. The process according to claim 3, wherein the compound A is a tertiary alcohol selected from tert-butanol, tert-amyl alcohol and pinacol.

17. The process according to claim 4, wherein the molar ratio ranges from about 2:1 to about 0.1:1.

18. The process according to claim 8, wherein Rₐ is an aromatic group selected from phenyl, tolyl, xylyl, mesityl, duryl, pentamethylphenyl, 2,6-diisopropylphenyl, tert-butylphenyl, ditertbutylphenyl, methoxyphenyl, dimethoxyphenyl, methoxytolyl, methylenedioxyphenyl, biphenyl, nitrophenyl, halogen substituted phenyl, trifluoromethylphenyl, naphtyl, pyridyl, furyl, pyrrolyl, thiophenyl, 2-indolyl, benzofuryl and all their position isomers.

19. The process according to claim 11, wherein the organic solvent is an aprotic solvent selected from tetrahydrofuran (THF), 2-methyltetrahydrofuran (MeTHF), toluene, xylene, anisole, diethyl ether, tert-butyl methyl ether (MTBE), dichloromethane (DCM), chloroform, dioxane, pentane, cyclopentane, hexane, cyclohexane, methylcyclohexane, benzene and acetonitrile.

20. The process according to claim 14, wherein step iii) is conducted in a non-aqueous solution.
